(19) United States
(12) Patent Application Publication
(10) Pub. No.: US 2015/0132709 A1
(43) Pub. Date: May 14, 2015

(54) TEETH WHITENER

(71) Applicant: AMOREPACIFIC CORPORATION, Seoul (KR)

(72) Inventors: Chang Man Park, Seoul (KR); Sungwon Yi, Seoul (KR); Junoh Kim, Seoul (KR); Chan Ki Lee, Seoul (KR); Chanho Kim, Seoul (KR); Youngeun Sung, Seoul (KR); Sangkyun Chae, Seoul (KR); Yu-jin Kim, Seoul (KR)

(21) Appl. No.: 14/400,710

(22) PCT Filed: May 14, 2013
(86) PCT No.: PCT/KR2013/004171

§ 371 Date: Nov. 12, 2014

(30) Foreign Application Priority Data
May 18, 2012 (KR) 10-2012-0052834

(51) Int. Cl. A61C 19/06

(52) U.S. Cl. CPC A61C 19/066; A61N 2005/0606
USPC 433/29

(57) ABSTRACT

Disclosed is a tooth whitening apparatus including: a mouthpiece to which a gel for applying hydrogen peroxide is applied; a lamp unit disposed at an inner tip of a grip case to whiten teeth by using light; a secondary battery disposed on a lower side of a PCB control board inside the grip case; a vibration device disposed at an upper side of the grip case to be operated by an on-off switch; and a USB port charging unit disposed at a rear side of the grip case to charge the secondary battery.

### CROSS REFERENCE RELATED APPLICATIONS

This application claims foreign priority of Korean Patent Application No. 10-2012-0052834, filed on May 18, 2012 and International Patent Application No. PCT/KR2013/004171 filed on May 14, 2013, which are incorporated by reference in their entirety into this application.

### BACKGROUND OF THE DISCLOSURE

1. Field of the Disclosure

The disclosure relates to a tooth whitening apparatus, and more particularly, to a tooth whitening apparatus which includes a vibration device in a body thereof to allow gel for applying hydrogen peroxide to be uniformly applied to teeth by using vibrations.

2. Description of the Prior Art

In recent years, a number of people pay attention to well being. Due to industrialization, people live well materially and now pay a lot of attention not to what they eat but to how they can live better lives. In this way, as they have a high standard of living, they try to change their outer appearances to make their lives more appealing. The tries include tooth whitening for making their teeth white, which can show a clear image to counterparts.

In the initial stage of tooth whitening, toothpaste containing a small amount of tooth whitening agent was used as a simple method for achieving a tooth whitening effect through brushing.

However, coloring ingredients deposited on surfaces of teeth cannot be effectively removed since an amount of tooth whitening agent contained in toothpaste is too small.

In order to whiten teeth more effectively, professional operations using tooth whitening pads or lasers are also used. This method is very effective in tooth whitening effect, the patient should go to hospital regularly for a long time and a high medical fee is required.

Accordingly, a tooth whitening patch has been proposed as an effective compromising measure which requires low costs and simple processing.

The tooth whitening patch is a product on which an ointment containing a tooth whitening agent based on carboxides or peroxides of a low concentration is applied, and shows an excellent tooth whitening effect since it contains a sufficient amount of tooth whitening ingredient and can be used for a long time. Further, since a treatment can be prepared only by attaching the patch on front and rear surfaces of teeth according to guidelines, the patch can be simply used by everyone.

However, since a portion of the tooth whitening patch which is attached to teeth is soluble in water, the patch may be separated from the original attaching location due to movements of muscles around the mouth of the user over time, in which case the ointment may stimulate or damage gums, tongues or oral mucosa.

Recently, tooth whitening apparatuses have been developed to supplement the problem. An example of such tooth whitening apparatuses is disclosed in Korean Utility Model No. 412016 in which a visible ray polymerizer body for teeth includes a case and a lamp holder, a lamp is disposed at a central portion of the lamp holder, and a separate mouthpiece for applying gel for whitening teeth and removing mouth odor or gel for applying hydrogen peroxide is provided.

According to the tooth whitening apparatus, gel for whitening teeth and removing mouth odor or gel for applying hydrogen peroxide is applied on the mouthpiece first, the mouthpiece is mounted on teeth, the lamp holder is positioned in front of the mouthpiece and then is bit by the mouth, and electric power is applied by pushing a power button of the body. Then, a visible ray is emitted from the lamp and is irradiated to the mouthpiece through the lamp holder to whiten teeth or apply hydrogen peroxide to the teeth.

However, a process of mounting the mouthpiece to which the gel for applying hydrogen peroxide on the teeth, biting the lamp holder with the mouth, and radiating a visible ray of the lamp is complex, and fluorine cannot be uniformly applied to the teeth only through the irradiation of the lamp.

In particular, if the battery for lighting up the lamp accommodated in the body is exhausted, the case should be separated and the battery should be replaced.

### SUMMARY OF THE DISCLOSURE

Accordingly, the disclosure has been made to solve the above-mentioned disadvantages of the prior art, and an object of the disclosure is to provide a tooth whitening apparatus in which a mouthpiece and a body are formed integrally so that a user can conveniently use the tooth whitening apparatus by applying gel for applying hydrogen peroxide to the mouthpiece and turning on a lamp immediately.

Another object of the disclosure is to provide a tooth whitening apparatus in which a vibration device is installed in a body so that gel for applying hydrogen peroxide is uniformly applied to teeth to further improve a tooth whitening effect.

Another object of the disclosure is to provide a tooth whitening apparatus in which a USB port charging unit is provided at a rear side of a body to charge a secondary battery in the body so that a case may not be separated from the body.

In accordance with one aspect of the disclosure, there is provided a tooth whitening apparatus including: a mouthpiece to which a gel for applying hydrogen peroxide is applied; a lamp unit disposed at an inner tip of a grip case to whiten teeth by using light; a secondary battery disposed on a lower side of a PCB control board inside the grip case; a vibration device disposed at an upper side of the grip case to be operated by an on-off switch; and a USB port charging unit disposed at a rear side of the grip case to charge the secondary battery.

According to the embodiments of the disclosure, a mouthpiece and a body are formed integrally so that a user can conveniently use the tooth whitening apparatus by applying gel for applying hydrogen peroxide to the mouthpiece and turning on a lamp immediately. Further, a vibration device is installed in a body so that gel for applying hydrogen peroxide is uniformly applied to teeth to further improve a tooth whitening effect. In addition, since a USB port charging unit is provided at a rear side of the body, a secondary battery disposed in the body can be immediately recharged after it is discharged.

### BRIEF DESCRIPTION OF THE DRAWINGS

The above and other objects, features and advantages of the disclosure will be more apparent from the following detailed description taken in conjunction with the accompanying drawings, in which:

FIG. 1 is a perspective view of a tooth whitening apparatus according to the disclosure;

FIG. 2 is a side sectional view of the tooth whitening apparatus according to the disclosure; and

FIG. 3 is a front view of the tooth whitening apparatus according to the disclosure.

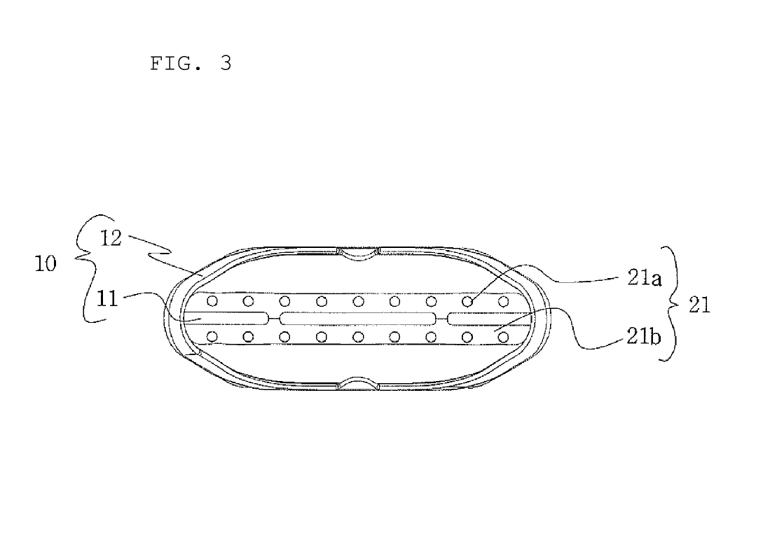

### DETAILED DESCRIPTION OF THE PREFERRED EMBODIMENTS

Hereinafter, an exemplary embodiment of the disclosure will be described in detail with reference to the accompanying drawings.

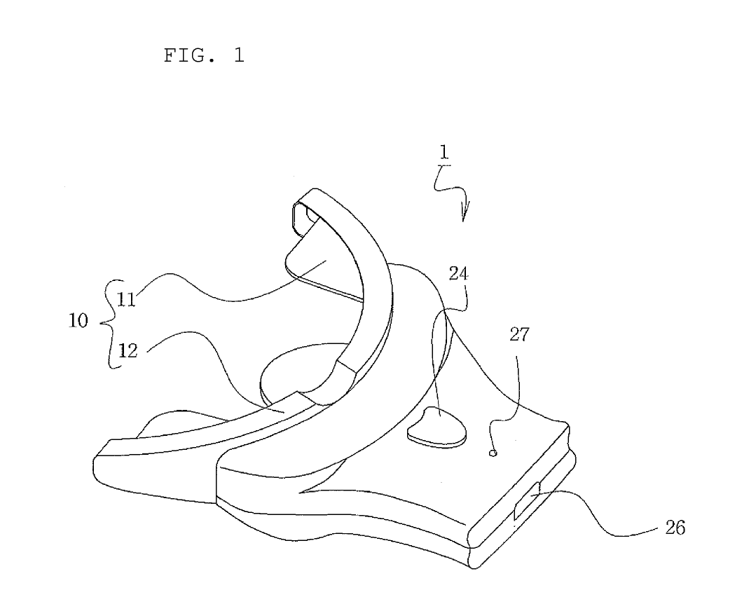

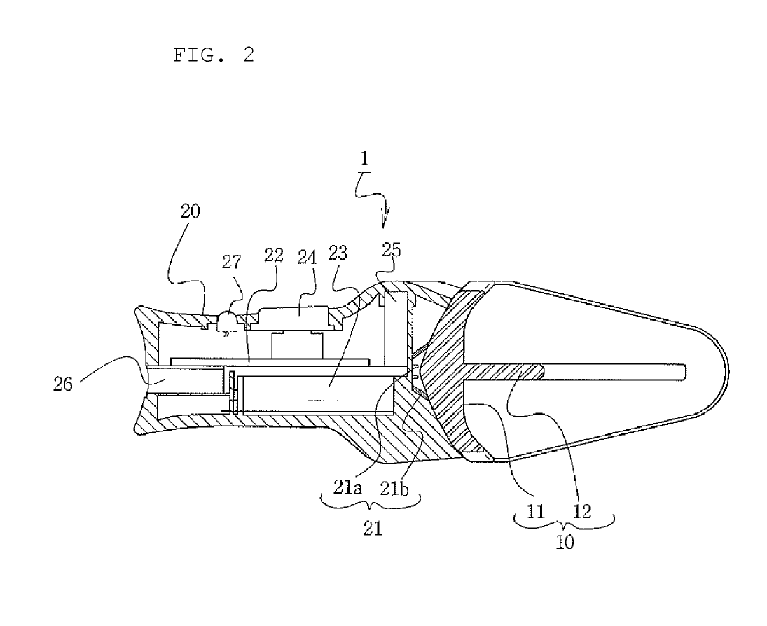

First, as shown in FIGS. 1 to 2, a body 1 for whitening teeth has a mouthpiece 10 to which a gel for applying hydrogen peroxide is applied; a lamp unit 21 disposed at an inner tip of a grip case 20 to whiten teeth by using light; and a secondary battery 23 disposed on a lower side of a PCB control board 22 inside the grip case 20; a vibration device 25 disposed at an upper side of the grip case 20 to be operated by an on-off switch 24; and a USB port charging unit 26 disposed at a rear side of the grip case 20 to charge the secondary battery 23.

Here, if the on-off switch 24 is switched on, a power lamp 27 disposed at an upper side of the grip case 20 lights up.

The mouth piece 10 includes a plurality of tooth bite plates 11 formed to be bit by teeth; and an arch plate 12 disposed at an inner side of the tooth bite plates 11 such that a tooth whitening agent surrounds front and rear surfaces of the teeth.

Here, the tooth bite plates 11 are transparent.

A plurality of lamps 21a are vertically disposed in parallel, and light guide plates 21b for expanding light are disposed on upper and lower sides of the lamps 21a.

The lamps 21a generate light of visible ray areas through LEDs.

An operation of the above-described tooth whitening apparatus according to the disclosure will be described below.

First, when the tooth whitening apparatus 1 is used, a user fills a tooth whitening agent on the tooth bite plates 11 of the mouthpiece 10, and bites the tooth bite plates 11 between upper and lower teeth. Then, the tooth whitening agent applied on the tooth bite plates 11 surrounds front and rear surfaces of teeth through the arch plate 12 disposed at a front side of the tooth whitening apparatus 1.

Accordingly, a whitening reaction of coloring ingredients deposited on teeth is started by tooth whitening ingredients contained in the tooth whitening agent.

If the on-off switch 24 is switched on in this state, the power lamp 27 lights up and the vibration device 25 is vibrated by a set program of the PCB control board 22 at the same time. Further, the lamps 21a provided in the lamp unit 21 light up.

Thus, if visible rays of the LEDs are emitted as a plurality of the lamps 21a light up, the whitening reaction of the whitening ingredients is promoted while photo active compounds contained in the tooth whitening agent is activated by light energy so that a whitening treatment time can be shortened, a whitening effect can be improved at the same time, and the whitening ingredients can be evenly applied to teeth by the vibration device 25.

Meanwhile, if the secondary battery 23 is discharged by the vibration device 25 and the lamps 21a, a power jack is plugged to the USB port charging unit 26 provided at a rear side of the grip case 20 to charge the secondary battery 23.

Although the disclosure has been described and shown with reference to the embodiment for exemplifying the principle of the disclosure, it is understood that the disclosure should not be limited to the above-described embodiment.

In addition, those skilled in the art can understand that various changes and modifications can be made therein without departing from the subject matters of the disclosure.

Thus, such changes and modifications fall within the spirit and scope of the disclosure.

What is claimed is:

1. A tooth whitening apparatus comprising:
a mouthpiece to which a gel for applying hydrogen peroxide is applied;
a lamp unit disposed at an inner tip of a grip case to whiten teeth by using light;
a secondary battery disposed on a lower side of a PCB control board inside the grip case;
a vibration device disposed at an upper side of the grip case to be operated by an on-off switch; and
a USB port charging unit disposed at a rear side of the grip case to charge the secondary battery.

2. The tooth whitening apparatus of claim 1, wherein the mouth piece comprises a plurality of tooth bite plates formed to be bit by teeth; and an arch plate disposed at an inner side of the tooth bite plates such that a tooth whitening agent surrounds front and rear surfaces of the teeth.

3. The tooth whitening apparatus of claim 1, wherein a plurality of lamps are vertically disposed in parallel, and light guide plates for expanding light are disposed on upper and lower sides of the lamps.

4. The tooth whitening apparatus of claim 3, wherein the lamps generate light of visible ray areas through LEDs.
